(19) United States
(12) Patent Application Publication
(10) Pub. No.: US 2006/0292700 A1
(43) Pub. Date: Dec. 28, 2006

(54) Diffused interrupted lateral flow immunoassay device and method

(76) Inventors: Naishu Wang, San Diego, CA (US); David F. Zhou, San Diego, CA (US)

Correspondence Address: CHARMASSON, BUCHACA & LEACH, LLP, 1545 HOTEL CIRCLE SOUTH, SUITE 150, SAN DIEGO, CA, 92108-3426, US

(21) Appl. No.: 11/158,547

(22) Filed: Jun. 22, 2005

(51) Int. Cl. G01N 33/558

(52) U.S. Cl. 436/514

(57) ABSTRACT

A lateral flow chromatographic immunoassay rapid test device having an additional interrupting, porous, diffusive pad pretreated with a surfactant interposed between the conjugate pad and the reaction membrane. The diffusive pad causes improved mixing of the fluid to provide a more mixture of molecules in the fluid and a more uniformly dispersed fluid flow having a more uniformly high degree of first affinity binding and a more even fluid front to cross into the reaction membrane, thereby increasing the sensitivity and specificity of the second affinity binding at both test and control lines, and thereby providing an important advancement of the existing lateral flow rapid test technology.

### FIELD OF THE INVENTION

This invention relates to chromatographic immunoassay test devices and more particularly to lateral flow rapid test devices used in a point-of-care setting.

### BACKGROUND

Lateral flow rapid tests based on the principles of chromatographic immunoassays have been used for many years for testing primarily body fluids such as whole blood, serum, plasma, urine, spinal fluid, amniotic fluid, mucous, saliva, and the like for the presence of infection or other conditions such as pregnancy, abused drugs and cardiovascular disorders such as acute myocardial infarction (AMI). They provide a convenient, quick, economic, and simplified way to conduct such tests without requiring sophisticated instrumentation or trained professionals. However, in many settings these rapid tests are useful only for preliminary screening purposes, not as a confirmatory test. To this day, for example, the Western Blot Analytical Assay is the only one reliably used for the confirmatory detection of HIV infection in a clinical laboratory setting worldwide. Due to its multi-step manipulation and verification phases, completion of this type of assay takes days, if not weeks. Such a delay can unfortunately lead to further propagation of infectious pathogens such as HIV or other serious results, such as the metastasis of cancers. There is virtually no generally accepted practical or economical confirmatory rapid diagnostic testing technique for use in a point-of-care setting to detect serious diseases such as HIV infection and AMI, available in the market place today.

Referring now to FIG. 1, there is shown a prior art lateral flow test device 1 which is typically constructed to have a sample pad 2 for accepting the fluid specimen which would carry at least one analyte, such as an antibody, which is specific to the condition being tested. The fluid specimen then flows through to a conjugate pad 3, in fluid flow contact with the sample pad. The conjugate pad is coated with at least one mobilizable binding member, such as an antigen (or antibody in some cases), which has immuno-determinant(s) (or specific binding sites for the immuno-determinant(s) in certain cases) of the analyte in question. The binding member is conjugated to a visibly detectable label such as colloidal gold. As the fluid passes through the conjugate pad, an immuno-chemical reaction occurs wherein the analyte in question binds to the binding member to form the first affinity binding labeled analyte complex. The fluid containing the labeled analyte complex then flows into one end of a reaction membrane 4, which is in fluid flow contact with the conjugate pad. The fluid is drawn primarily by capillary forces through the membrane toward an absorbant pad 5 which is in fluid flow contact with the opposite end of the membrane. The membrane is coated in a first test zone 6 with at least one immobilized capture binding member which is similarly immuno-determinant of the analyte in question. As the fluid passes through the membrane, a second immuno-chemical reaction occurs wherein the labeled analyte complex binds to the capture binding member to form the second affinity binding immuno-sandwich complex. The accumulation of the secondly bound immuno-sandwich complex beyond a threshold amount in the first zone 6 creates a colored test line, the so-called “T-line”. A second zone 7 located further downstream from the T-line is provided as an internal system control line, the so-called “C-line”. The control line is used as an indicator of functional validity. A protective plastic tape cover 8 is placed over the strip extending from the end of the sample pad 2 to the beginning of reaction membrane 4.

Unfortunately, depending on the type of condition being detected, these tests provide a typical accuracy of between 85% and 99%, falling short of the 99.5% or above accuracy generally considered to be necessary for a confirmatory test. The reasons for the insufficient accuracy are primarily due to the lack of overall higher sensitivity and specificity of the device. Different samples may contain chemicals or particles which interfere with or inhibit the fluid flow or otherwise interfere with one or both of the affinity binding reactions. Prior devices have attempted to enhance sensitivity or specificity by pretreating various parts of the devices with reaction or flow enhancing reagents, pH conditioning chemicals, or even precoating with non-specific adhesive blocking molecules which will “block-out” non-analyte molecules which might cause non-specific adhesion, or otherwise compete with the analyte in question for specific binding members, especially on the reaction membrane. These attempts have met with limited success in some types of testing, but do not provide the desired accuracy in many others. Also, pretreatment with two or more of the above pretreatments exacerbates the difficulties in obtaining uniform manufacturing due to potential incompatibilities between the pretreatment chemicals. For example, the pH conditioner might disrupt the effectiveness of the non-specific blocking member molecules. Or, the manufacturing step of pretreating with the second pretreatment chemical can dislodge some of the first pretreatment chemical.

Further, lot-to-lot variation in the manufacture of chromatographic strips, including non-uniform thicknesses and densities of the pad material, non-uniform coating of the binding members or non-uniform pretreatment of any reagents, pH conditioners, non-specific adhesive blocking molecules can often lead to ambiguous results, such as false negatives as well as weak false positives, so-called “ghost lines” or “phantom lines”. False negatives typically occur when non-specific molecules interfere with the first and/or second affinity binding actions. It has been found that non-analyte molecules can clump together in fluid samples that are not well mixed so that they temporarily prevent access between analytes and binding members. Even temporary interference can prevent an adequate number of labeled analyte complexes and/or ulitmately immuno-sandwich complexes from forming. It is believed that this is due to most complexes being formed at the leading edge or very close to the leading edge or fluid front of the fluid flow through lateral flow devices. In this way, if a non-analyte molecule or clump of molecules blocks access between analytes and binding members for only a few seconds, it may be enough to induce a false negative result. Further, clumps of non-analyte molecules can carry an overabundance of the labeled mobilizable binding members to the second affinity binding site to generate a false positive.

Referring now to FIG. 2, there is shown a top view of a stylized chromatographic test strip 10 according to the prior art having a length L direction and a width W direction. The strip has a porous sample pad 11, adjacent to a porous conjugate pad 12, which is adjacent to a reaction membrane 13 having T-line 14 and a C-line 15 zones. The conjugate pad 12 has been coated with a now dried reagent containing a mobilizable, labeled binding member for the analyte in question. Due to manufacturing inconsistencies, there can exist variations in the concentration of the dried reagent as indicated for example by the regions 20,21,22 of the conjugate pad. Region 20 shows the weakest concentration, while region 22 shows greatest concentration. A fluid specimen 24 is deposited on the sample pad 11 and begins flowing 25 laterally, mainly through capillary forces, toward the conjugate pad 12. Within the specimen there can be portions wherein the concentration of analyte molecules is greater and other portions where the concentration of non-analyte molecules is greater, or even where such non-analyte molecules have clumped together. Because of other manufacturing inconsistencies, the density of the pads is not uniform, leading to differences in flow rate at various locations of the pads. These differences are indicated by differently sized arrows 27,28,29. The smallest arrow 27 shows the weakest flow, whereas the largest arrow 29 shows the strongest flow. This creates an uneven fluid front or leading edge of the flow 23. In this example, differential and non-chemically uniform flows exist across the width and length of the conjugate pad resulting in flows exiting the conjugate pad having non-uniform first affinity binding. Some flows 30,31 have flowed more quickly than others to reach the dried reagent first, and/or flowed through regions of higher concentrations of dried reagent, and/or carry a greater concentration of clumps of non-analyte molecules which can carry away labeled mobilized binding members. These flows 30,31 exhibit a greater degree of first affinity binding per unit fluid or at least uptake of mobilizable labeled binding members. The darkness of the shading of the flow arrows indicates the degree of apparent first affinity binding. Other flows 32,35 having flowed more slowly and/or through a region of lower reagent concentration 20 and/or having a lower than average concentration of analyte molecules, and/or having a greater concentration of non-clumped, non-analyte molecules which merely inhibit analyte binding but do not carry away mobilizable labeled binding members, exhibit less apparent first affinity binding. Additionally, the differential flow creates an uneven fluid front 33. Because the leading edge of the fluid front often carries the highest concentration of labeled binding members, it is preferable that the liquid front be even or have a more planar shape which is substantially parallel to the T and C lines zones, which in this example would be a vertical line 34, indicating arrival of the fluid across substantially the entire width the T and C line zones at substantially the same time. These flow and concentration dis-uniformities are responsible for many of the unsatisfactory results discussed above.

Therefore, there is a need to refine the accuracy of chromatographic lateral flow rapid testing devices for the purpose of use in early confirmatory point-of-care diagnostic setting.

### SUMMARY

The instant embodiments provide an advanced, more efficient and more discriminative way of rapidly detecting the presence of cancer, infection or other conditions such as pregnancy, cardiovascular disorders, and abused drugs in body fluids through the use of lateral flow chromatographic immunoassay test devices and thus potentially avoiding the long turn-around time required by use of separate assays using multiple tests such as in a Western Blot assay, or other sophisticated testing methods to achieve a confirmatory result.

Some embodiments provide an interrupting, porous, diffusive pad interposed between the conjugate pad and the reaction membrane. The diffusive pad causes improved mixing of the fluid to provide a more uniformly dispersed fluid flow having a more uniform mixture of molecules, and thus a more uniformly high degree of first affinity binding before crossing into the reaction membrane and thereby enhancing the quality of the second affinity binding which results in an increase in the sensitivity and specificity (accuracy) of lateral flow testing devices, and constitutes an advancement in performance over prior lateral flow testing devices.

Some embodiments provide that in a lateral flow chromatographic test apparatus comprising a conjugate pad adapted to carry a soluble binding member molecule specific to a particular analyte and a reaction membrane carrying a capture binding member molecule, there is an improvement which comprises: a first porous interrupting diffusive pad located between a portion of said conjugate pad and a portion of said reaction membrane, thereby creating a plurality of convergent fluid paths between said conjugate pad and said membrane. In some embodiments the portion of said conjugate pad overlaps said first diffusive pad, and a portion of said first diffusive pad overlaps said portion of said reaction membrane. In some embodiments said first diffusive pad is pretreated with a surfactant. In some embodiments said surfactant comprises a non-ionic detergent. In some embodiments said first diffusive pad has a first pretreatment condition. In some embodiments said first pretreatment condition is being pretreated with a surfactant. In some embodiments said first pretreatment condition is selected from the group consisting of: being pretreated with a surfactant; being pretreated with a pH conditioner, being pretreated with a non-specific adhesive blocking molecule, and having no pretreatment. In some embodiments said improvement further comprises a second diffusive pad, wherein said first diffusive pad has a first pretreatment condition and said second diffusive pad has a second pretreatment condition, and wherein said first pretreatment condition is different from said second pretreatment condition. In some embodiments said first pretreatment condition is selected from the group consisting of: being pretreated with a surfactant; being pretreated with a pH conditioner, being pretreated with a non-specific adhesive blocking molecule, and having no pretreatment.

Some embodiments provide that the diffusive pad is made from material having a plurality of intersecting surface structures, wherein a junction between first and second of said structures is not substantially parallel. In some embodiments said diffusive pad comprises a material selected from the group consisting of fiberglass, cellulose and fibrous plastic and the like. In some embodiments said diffusive pad comprises means for evening a fluid front passing therethrough. In some embodiments there is no direct fluid flow contact between said conjugate pad and said reaction membrane.

Some embodiments provide a method for conducting a lateral flow immunoassay test which comprises diffusing a fluid flow from a conjugate pad through a diffusive pad before said flow reaches a reaction membrane.

Some embodiments provide a method for detecting a biological condition by means of a lateral flow chromatographic immunoassay rapid test on a sample of a patient's body fluid which comprises: exposing said sample to a strip having at least one line carrying an immunoassay complex component responsive to at least one inter-reactive component indicative of the presence of said condition in said patient; mixing said sample after said exposing, using a diffusive pad, to form a mixture; and, distributing said mixture to a reaction membrane which carries said at least one line. In some embodiments said mixing comprises evening a fluid front within said diffusive pad. Some embodiments further comprise pretreating said diffusive pad with a surfactant. In some embodiments said detecting occurs at an accuracy of at least 99.5%.

Some embodiments provide an immunochromatographic assay device for the detection of the presence or absence of analyte in a liquid sample comprises: a sample pad comprising a porous material; a conjugate pad comprising a porous material and at least one mobilizable labeled reagent capable of binding an analyte in question and forming a labeled reagent-analyte complex; a diffusive interrupting pad comprising a porous material; a reaction membrane comprising a porous material and at least one immobilized capture reagent capable of binding said labeled reagent-analyte complex; and, an absorbent pad comprising a porous material which conducts fluid flow of said liquid sample and is capable of absorbing excess liquid sample; wherein said sample pad is in fluid flow contact with said conjugate pad, and said conjugate pad is in fluid flow contact with said diffusive pad, and said diffusive pad is in fluid flow contact with said reaction membrane, and said reaction membrane is in fluid flow contact with said absorbent pad; and, wherein the liquid sample flows from the sample pad to the absorbent pad during an operation time. In some embodiments said diffusive pad comprises fiberglass. In some embodiments said diffusive pad further comprises a chemical to increase specific binding of the labeled reagent-analyte complex. In some embodiments said fluid flow contact is by overlap. In some embodiments said overlap is 50-75%. In some embodiments said diffusive pad provides a fluid flow delay between about 2 and 3 seconds. In some embodiments the device further comprises a cover over said conjugate pad and said diffusive pad.

Some embodiments provide an immunochromatographic assay device for the detection of the presence or absence of analyte in a liquid sample comprising: means for delaying fluid flow from a conjugate pad to a reaction membrane. In some embodiments said means for delaying fluid flow comprises a separate porous material placed between said conjugate pad and said reaction membrane. In some embodiments said porous material comprises fiberglass. In some embodiments said means for delaying fluid flow further comprises a chemical to increase specific binding of a labeled reagent and the analyte. In some embodiments said conjugate pad, reaction membrane and means for delaying fluid flow are in fluid flow contact. In some embodiments said fluid flow contact is by overlap. In some embodiments said overlap is 50-75%. In some embodiments said means for delaying provides a delay of between about 2 and 3 seconds.

Some embodiments provide a method for detecting the presence or absence of analyte in a liquid sample comprising applying the liquid sample to the sample pad of the device of an earlier embodiment and detecting the presence or absence of analyte at the reaction membrane, with an accuracy of at least 99.5%.

Some embodiments provide an immunochromatographic assay device for the detection of the presence or absence of analyte in a liquid sample comprising means for premixing said sample prior to exposure to a conjugate pad.

### BRIEF DESCRIPTION OF THE DRAWING

FIG. 1 is a diagrammatical cross sectional side view illustration of a prior art chromatographic lateral flow test strip.

FIG. 2 is a diagrammatical top view illustration of a test strip of the prior art showing flow volume and first affinity binding concentration dis-uniformities.

FIG. 3 is a diagrammatical cross-sectional side view illustration of a test strip having a diffusive, flow interrupting pad.

FIG. 4 is a diagrammatical top view illustration of a test strip having a diffusive, flow interrupting pad and showing flow volume and conjugate concentration uniformization.

FIG. 5 is a diagrammatical microscopic illustration of fibers in a diffusive, flow interrupting pad material.

FIG. 6 is a diagrammatical microscopic close-up illustration of a fiber junction of a diffusive, flow interrupting pad material of FIG. 5 taken at box 6-6.

FIG. 7 is a diagrammatical microscopic illustration of the fiber junction of FIG. 6 where microscopic fluid flows are converging.

FIG. 8 is a diagrammatical microscopic illustration of the fiber junction of FIG. 6 where microscopic fluid flows have converged and mixed.

FIG. 9 is a diagrammatical cross-sectional close-up side view illustration of test strip of FIG. 3 showing fluid flows into the dry portion of the diffusive pad.

FIG. 10 is a diagrammatical cross-sectional close-up side view illustration of test strip of FIG. 3 showing downward fluid flows across the diffusive pad/reaction membrane border.

FIG. 11 is a diagrammatical cross-sectional side view illustration of a test strip having two differently pretreated diffusive, flow interrupting pads straddling the conjugate pad.

FIG. 12 is a diagrammatical cross-sectional side view illustration of a test strip having two differently pretreated diffusive, flow interrupting pads downstream of the conjugate pad.

FIG. 13 is a diagrammatical side view illustration of a test strip using the diffusive pad and manufactured using a simple, partially overlapping pad structure.

FIG. 14 is a diagrammatical exploded perspective view illustration of the arrangement order of different component pads of a test strip according to FIG. 13.

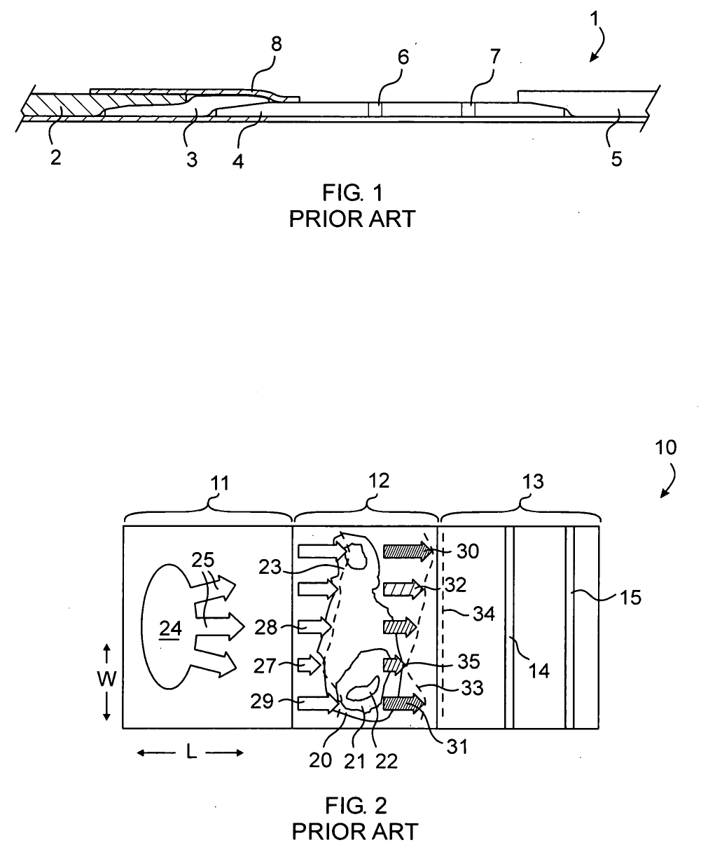

### DESCRIPTION OF THE PREFERRED EMBODIMENTS

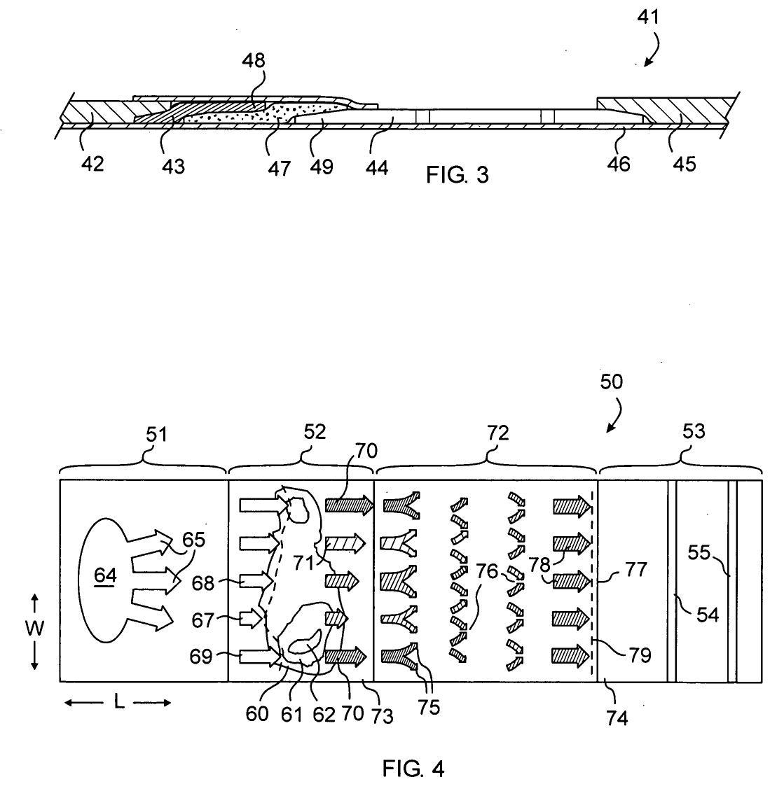

Referring now to FIG. 3, as a first embodiment, there is shown a test strip 41 adapted for use on a lateral flow rapid chromatographic immunoassay test device which comprises a porous sample pad 42, adjacent to a porous conjugate pad 43 coated with a now dried reagent containing a mobilizable, labeled binding member for the analyte in question. Those pads adjacent to one another are said to be in fluid flow contact with one another. The strip has a reaction membrane 44, and an absorption pad 45, mounted atop a fluid impermeable vinyl backing 46. A diffusive pad 47 is mounted between a portion 48 of the conjugate pad and a portion 49 of the reaction membrane thereby separating the conjugate pad and reaction membrane from one another.

Referring now to FIG. 4, there is shown a top view of a stylized chromatographic test strip 50 similar to the strip of FIG. 2 in that it has a length direction L and a width direction W, a porous sample pad 51, adjacent to a porous conjugate pad 52, and a reaction membrane 53 having T-line 54 and a C-line 55 zones. Again similarly, the conjugate pad 52 has been coated with a now dried reagent containing a mobilizable, labeled binding member for the analyte in question. Due to technical limitations and manufacturing inconsistencies, there exists variation in the concentration of the dried reagent as indicated by the regions 60,61,62 of the conjugate pad. Region 60 shows the weakest concentration, while region 62 shows greatest concentration. A fluid specimen 64 having chemical dis-uniformities is deposited on the sample pad 51 and begins flowing 65 laterally toward the conjugate pad 52. Because of other manufacturing inconsistencies, the density of the particles in the pads is not uniform, leading to differences in flow rate at various locations of the conjugate pad. These differences are indicated by differently sized arrows 67,68,69. The smallest arrow 67 shows the weakest flow, whereas the largest arrow 69 shows the strongest flow. In this example, these differential flows exist across the width and length of the conjugate pad. These conditions result in some flows 70 exhibiting a greater apparent degree of first affinity binding per unit fluid, whereas other flows 71 exhibit a lesser apparent degree of first affinity binding. The darkness of the shading of the flow arrows schematically indicates the degree of apparent first affinity binding.

The primary difference between the strip of FIG. 4 and the strip of FIG. 2 is that the strip of FIG. 4 includes a diffusive, interrupting pad 72 located between a portion 73 of the conjugate pad 52 and a portion 74 of the reaction membrane 53 thereby separating the conjugate pad and reaction membrane from one another. In this way there is no direct fluid flow contact between the conjugate pad and the reaction membrane, but rather the fluid flow must pass through the diffusive pad before reaching the reaction membrane.

The function of the diffusive pad 72 of the flow of fluid is primarily three-fold. Firstly, the porous diffusive pad acts to cause the flow to separate at the fluid front into a plurality of branches or furcations 75 which, when these furcations converge and join together again, do so from different directions. The convergence from different directions 76 causes a mixing across the fluid front and hence the entire specimen as it flows forward. This mixing can cause the break-up of clumps of non-analyte molecules which may carry mobilizable labeled binding members, to reduce false positives. The mixing also reduces the differences in the concentrations of non-analyte molecules and labeled analyte complexes so that they are spread more evenly. Once the fluid front reaches the far end 77 of the diffusive pad, the concentrations have superior uniformity as indicated by the similar shading of the flow arrows 78. This uniformity leads directly to giving the labeled analyte complexes a greater opportunity to form the second affinity binding and thereby reducing false negatives.

Secondly, the diffusive action of the diffusive pad 72 automatically delays or interrupts the flow from the conjugate pad 52 to the reaction membrane 53 providing more incubation time for the first affinity binding to occur, thereby increasing the overall sensitivity and specificity of the test.

Thirdly, the diffusive action of the diffusive pad causes the fluid front 79 to be more even or planar in shape so that the fluid specimen enters the reaction membrane 53 more evenly across the width of its first end 74, substantially parallel with the second affinity binding zones 54,55 and tends to arrive at the second affinity binding zones simultaneously across the width of the device. In this way the diffuse pad provides a means for evening the fluid front passing therethrough.

Referring now to FIGS. 5-8, the diffusive pad is preferably made from relatively non-reactive, porous material 80 such as fiberglass, cellulose, polysullfone, NYLON brand material, polyethylene, NOVYLON brand material, POREX CHEMISTRY K brand material, POREX CHEMISTRY A brand material, FILTRONA brand material, and the like, all commercially available. The material is selected to have intersecting surface structures such as fibers 81 oriented substantially differently to one another to create a plurality of junctions 82 where there is typically an angle Aⱼ formed between the two intersecting structures at their junction which is not 0 degrees. In other words, at the junction, the fibers should not be substantially parallel so that they provide intersecting surfaces which, through surface tension forces cause the fluid to branch into furcations and converge causing a more thorough intermixing. It is important to note that the viscosity of the fluid can be a factor in determining the optimum density of fibers and hence the number of junctions required in a given volume of material. However, it has been found that fiberglass material commercially available from JBC of Elyria, Ohio provides an adequate number of junctions for most applications. Other non-fibrous porous materials can be used that provide intersecting surface structures to cause fluid furcation and intermixing, and are preferably capable of carrying a dried surfactant.

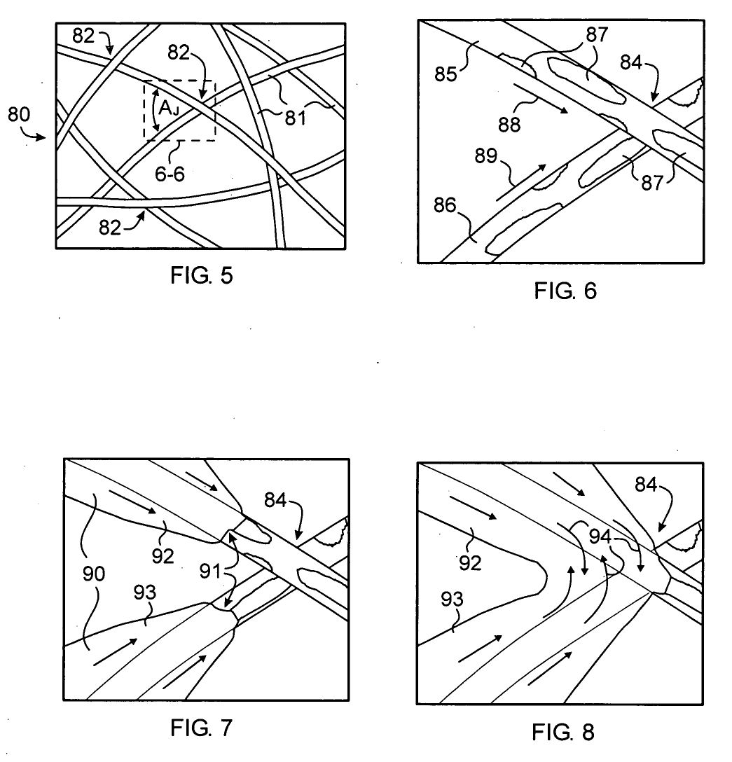

The diffusive pad is pretreated with a surfactant by immersing the pad into an amount of liquid surfactant so that it penetrates substantially all of the pores of the pad. The pad is then dried. This leaves a residue of the surfactant on the intersecting surface structures. As shown in FIG. 6, the junction 84 of two intersecting surface structures such as fibers 85,86 having dried surfactant residue 87 thereon creates two convergent fluid paths 88,89. As shown in FIG. 7, fluid 90 flowing through the diffusive pad will at its fluid front 91 have an affinity for separating into branches or furcations 92,93 which each tend to flow along the fluid paths formed by each surfactant treated fiber. As shown in FIG. 8, the furcations 92,93 will meet at the fiber junction 84 and their respective velocities will cause an intermixing as indicated by flow lines 94.

The preferred surfactant is a detergent such as polyethylene glycol sorbitan monolaurate commercially available under the brand name TWEEN 20 from Sigma-Aldrich Corporation of St. Louis, Missouri. Other detergents are acceptable such as TRITON X-100 brand, TRITON X-114 brand, TWEEN 80 brand, and sodium dodecyl sulfate (“SDS”) detergents also available from Sigma-Aldrich Corporation. Depending on the test being conducted, other anionic, cationic, non-ionic and Zwitterionic detergents may be acceptable.

It should be noted that the surfactant in the dry portion of the diffusive pad naturally provides, through capillary surface tension forces, a greater affinity for fluid to flow further into the diffusive pad rather than into the adjacent downstream structure such as the reaction membrane. In this way, the surfactant treated diffusive pad provides additional functions to increase uniformity of the fluid, to cause a more even fluid front before allowing the fluid to cross into the reaction membrane, which enhances the sensitivity and specificity of the affinity bindings as described below.

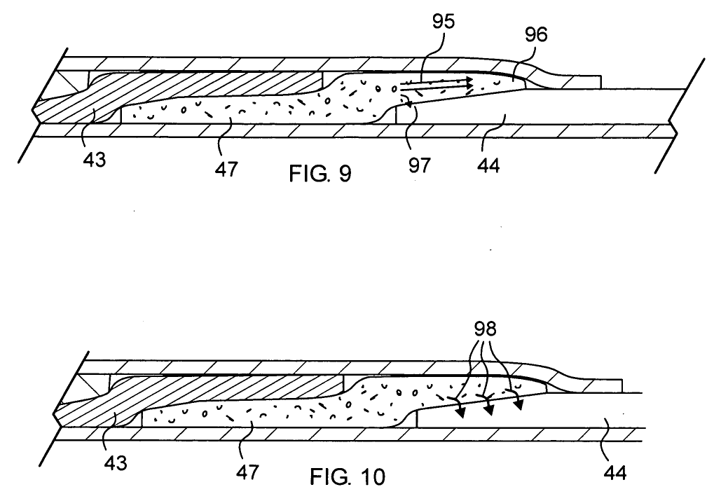

Firstly, referring now to FIG. 9, the fluid flow being driven primarily through capillary forces will tend toward fully saturating the diffusive pad before any substantial flow leaves the diffusive pad 47 for the reaction membrane 44. This preferential fluid flow direction is primarily due to the differential surface tension caused by the surfactant treatment of the structures in the diffusive pad. Arrows 95 indicate that the predominant flow will be first toward the dry, downstream portion 96 of the diffusive pad rather than across the border 97 between the diffusive pad and the reaction membrane 44. In this way the diffusive pad acts partially as a reservoir for delaying or interrupting the flow into the reaction membrane giving more time for first affinity binding to occur. Depending on the chemistry of the test being performed and the viscosity of the sample being tested, the flow can be delayed between about 1 and about 40 seconds. For relatively low viscosity samples such as urine, the flow is delayed for between about 2 and about 3 seconds.

Secondly, referring now to FIG. 10, as the diffusive pad 47 eventually becomes saturated and the fluid pressure, due to continued capillary action, builds at the border, the fluid front will then rush across the border into the reaction membrane 44 along an even fluid front. This relatively rapid breakdown of the surface tension barrier at the border causes further mixing and leads to a more even fluid front. Further, because of the overlapping structure of the diffusive pad 47 to the reaction membrane 44, the direction of flow of the fluid front must make a downward turn 98 to flow into the reaction membrane. This change in direction also serves to better mix the fluid. Therefore, to cause this downward turn, the border 97 between the diffusive pad and the reaction membrane should preferably be an overlapping border. The overlap can be, for example, 25, 30, 40, 50, 60, 75, or even more than 80% of the length of the diffusive pad.

Pretreatment of the diffusive pad can also be in the form of pH conditioning chemicals, and non-specific adhesive blocking molecules which will selectively filter or “block-out” unwanted non-analyte adhesion molecules, typically proteins, which can non-specifically interfere and/or compete with the analyte in question in either or both of the first and second affinity binding stages of the lateral flow test.

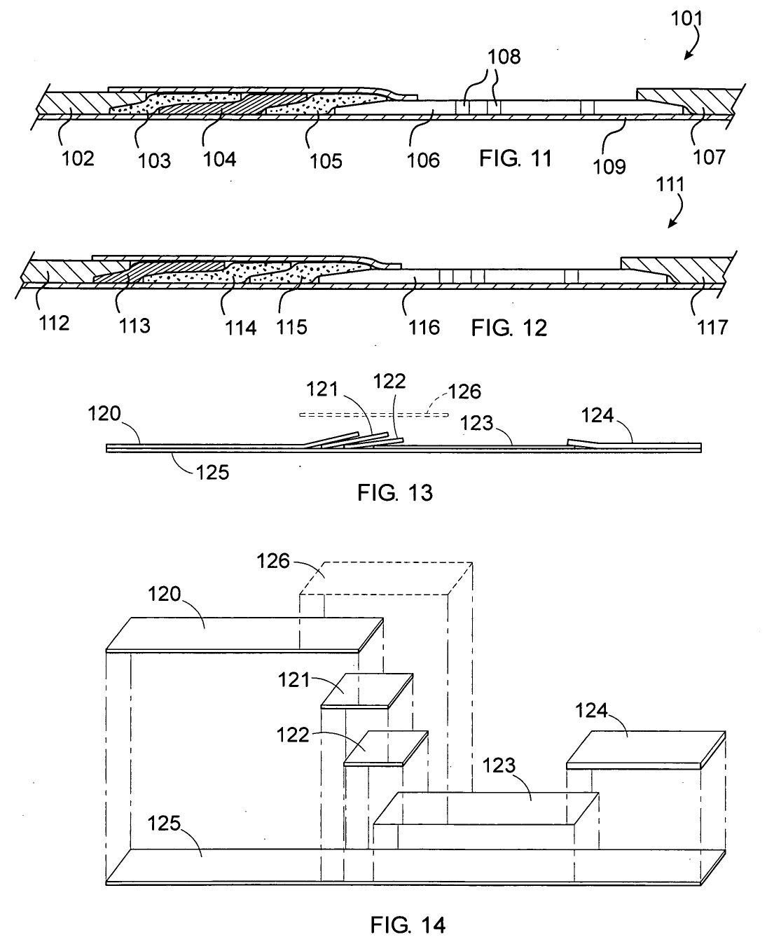

Referring now to FIG. 11 there is shown a test strip 101 adapted for use on a lateral flow rapid chromatographic immunoassay test device having a porous sample pad 102 adjacent to a first, porous upstream diffusive pad 103, which is adjacent to a conjugate pad 104 coated with a dried reagent containing a mobilizable, labeled binding member for the analyte in question. The diffusive pad being upstream from the conjugate pad will help to premix the fluid specimen to provide for more accurate first affinity binding to occur, and potentially to break up clumping of non-analyte molecules which can lead to false mobilization of labeled binding members. The conjugate pad is also adjacent to a second, downstream diffusive pad 105 which is adjacent to the upstream end of a reaction membrane 106 which is adjacent at its downstream end to an absorption pad 107. These pad structures are secured atop a fluid impermeable plastic backing 109. Those pads adjacent to one another are said to be in fluid flow contact with one another. In this embodiment the two diffusive pads 103,105 straddle the conjugate pad 104.

The first diffusive pad may be pretreated with a dried surfactant similar to one of the above embodiments. In addition to filtering out larger particles in the fluid sample, the porous first diffusive pad would then provide a more even fluid front to the conjugate pad, helping provide a more uniform first affinity binding even as the fluid front passes out of the conjugate pad.

In another embodiment the first, upstream diffusive pad can have a pretreatment condition which includes a pH conditioning chemical, also in a dried form, which can change the pH of the fluid sample to a more optimal range for first affinity binding to occur. Similarly, the first diffusive pad 103 can also be pretreated to include non-specific adhesive blocking molecules to further filter out undesirable molecules prior to first affinity binding. Similar to the embodiment of FIG. 3, the second pad 105, downstream from the conjugate pad 104 would have a pretreatment condition of having a dried surfactant therein.

It should be noted that the increased accuracy provides the ability to make strips having multiple “T-line” zones 108 for detecting different analytes in a single specimen such as for detecting multiple major epitopes, i.e., antigenic determinants of a single pathogen, or pathogenic organism such as bacteria, virus, parasites, rickettsia, and the like, with higher sensitivity and specificity compared to that of prior devices. It can also have multiple “T-lines” for detecting a set of bio-medical markers such as Troponin I, CK-MB, CRP and myoglobin for an endogenious disorder such as AMI with higher accuracy performance.

Referring now to FIG. 12 there is shown another alternate embodiment wherein a test strip 111 has a porous sample pad 112 adjacent to a conjugate pad 113 coated with a dried reagent containing a mobilizable, labeled binding member for the analyte in question. The conjugate pad is adjacent to a first, porous upstream diffusive pad 114, which is adjacent to second, downstream diffusive pad 115 which is adjacent to the upstream end of a reaction membrane 116 which is adjacent at its downstream end to an absorption pad 117. Those pads adjacent to one another are said to be in fluid flow contact with one another. In this embodiment the upstream diffusive pad 114 can have a different pretreatment condition than the downstream diffusive pad 115. For example, the upstream pad can be pretreated to have a pH conditioner; and the downstream pad be pretreated to have a non-specific adhesive blocking molecule and a surfactant.

By splitting the pretreatment chemicals between two or more pads, the strip designer is not only given increased accuracy, but also more control and predictability over the reactions occurring at any given stage of the lateral flow process. Also, using two or more pretreated pads potentially avoids manufacturing inconsistencies such as the second pretreatment chemical dislodging some of the first pretreatment chemical.

It should be noted that in either of the multi-diffusive pad embodiments, no pretreatment can be a pretreatment condition. For example, the upstream pad 103 in FIG. 11 may simply be a fiberglass filter pad without any surfactant, pH conditioner or non-specific adhesive blocking molecule. In this case its pretreatment condition would be “unpretreated”. This pretreatment condition would be different from the pretreatment condition of the downstream pad 105 which could have a dried surfactant pretreatment condition.

Again it should be noted that the increased accuracy provides the ability to make multi-line strips for detecting different analytes in a single specimen such as for detecting multiple major epitopes, i.e., antigenic determinants of an endogenious disorder such as AMI, or a single exogenous pathogen, or pathogenic organism such as bacteria, viruses such as HIV, parasites, rickettsia, and the like.

Referring now to FIGS. 13 and 14, the strip can be formed using a simplified manufacturing process which merely overlaps the individual porous material elements or pads. For example, a series of separated elements such as a sample pad 120, a conjugate pad 121, a diffusive interrupting pad 122, a reaction membrane 123, and an absorption pad 124, are attached to an elongated backing strip of a semi-rigid, fluid impermeable material 125, such as vinyl and the like. A plastic tab (or tape) 126 is optionally used to protect the conjugate pad, and optionally to cover a portion of the diffusive pad, the reaction membrane and the sample pad. The sample pad, conjugate pad, diffusive pad, reaction membrane, and absorption pad are in direct lateral fluid flow contact with each other such that the direction of fluid flow in the test device is from the sample pad laterally (i.e., by capillary force or wicking action) toward the end of the absorption pad. As shown in FIG. 14, each pad may be laminarly and overlappingly placed upon the backing and secured in place to form the strip.

Depending on the disease being tested and the condition of the fluid specimen, many of the above embodiments have been found to achieve an accuracy of at least 99.5%.

The terms “sample” and “specimen” as used herein refers to any biological sample that could contain an analyte for detection. The biological sample can be in liquid form or can be changed into a liquid form. The sample can include, for example, body fluids such as whole blood, serum, plasma, urine, spinal fluid, amniotic fluid, mucous, saliva, and the like.

The term “analyte” as used herein refers to a molecule, a compound or a composition to be detected or measured in the test sample. The analyte can be any substance for which there exists a naturally occurring specific binding member (e.g., an antibody) or for which a specific binding member can be prepared immunologically. The analyte will have at least one epitope that an antibody or an immunological reactive fragment thereof can recognize and bind to. Analyte can include any antigenic substances, haptens, antibodies and combinations thereof. The analyte of interest in an assay can be, for example, a protein, a peptide, an amino acid, a ligand, a nucleic acid, a hormone, asteroid, a vitamin, a pathogenic microorganism or even a synthetic peptide as well as its genetically engineered recombinant proteins for which polyclonal and/or monoclonal antibodies can be produced, a natural or synthetic chemical substance, a contaminant, a drug including those administered for therapeutic purposes as well as those administered for illicit purposes, and metabolites of or antibodies to any of the above substances. The analyte can also comprise an antigenic marker or antibody for single or multiple pathogenic conditions.

Representative analytes include steroids such as estrone, estradiol, cortisol, testosterone, progesterone, chenodeoxycholic acid, digoxin, cholic acid, digitoxin, deoxycholic acid, lithocholic acids and the ester and amide derivatives thereof; vitamins such as B-12, folic acid, thyroxine, triiodothyronine, histamine, serotonin, prostaglandins such as PGE, PGF, PGA; antiasthmatic drugs such as theophylline, antineoplastic drugs such as doxorubicin and methotrexate; antiarrhythmic drugs such as disopyramide, lidocaine, procainamide, propranolol, quinidine, N-acetylprocainamide; anticonvulsant drugs such as phenobarbital, phenytoin, primidone, valproic acid, carbamazepine and ethosuximide; antibiotics such as penicillins, cephalosporins, erythromycin, vancomycin, gentamicin, amikacin, chloramphenicol, streptomycin and tobramycin; antiarthritic drugs such as salicylate; antidepressant drugs including tricyclics such as nortriptyline, amitriptyline, imipramine and desipramine; as well as metabolites thereof. Additional therapeutic drugs include, for example, carbamazepine, free carbamazepine, cyclosporine, digoxin, FK778, gentamicin, lithium, N-acetylprocainamide, quinidine, tacrolimus, valproic acid, free valproic acid, and the like, as well as the metabolites thereof.

Representative analytes also include drugs of abuse, and their metabolities, including amphetamines, methamphetamines, barbiturates, benzodiazepines (BZD), cannabinoids, cocaine (benzoylecgonine), opiates, phencyclidine (PCP), tricyclic antidepressants (TCA), methadone, propoxyphene (PPX), marijuana (THC), methylenedioxymethamphetamine (MDMA, or Ecstasy, or XTC), morphine, oxycodone, and bupromorphine. Representative drugs of abuse include ethanol, heroin, hydromophone, oxymorphone, metapon, codeine, hydrocodone, dihydrocodiene, dihydrohydroxy codeinone, pholcodine, dextromethorphan, phenazocine and deonin.

Hepatic analytes include, for example, albumin bromocresol green (BCG) or purple (BCP), alkaline phosphatase, hepatitis B core antigen antibody (anti-HBc), hepatitis B e antigen antibody (anti-HBe), hepatitis B surface antigen antibody (anti-HBs), hepatitis C virus (HCV), anti-HCV, direct bilirubin, gamma-glutamyl transpeptidase (GGT), antibody to Hepatitis A virus (HAVAb)—IgG, HAVAb—IgM, hepatitis B surface antigen (HBsAg), lactate dehydrogenase (LD), neonatal bilirubin, prealbumin, total bilirubin, total protein, and the like.

Analytes related to pregnancy and fertility include, for example, human chorionic gonadotropin (hCG), beta-hCG, total beta-hCG, luteinizing hormone (LH), follicle stimulating hormone (FSH), dehydroepiandrosterone sulfate (DHEAS), estradiol, free estriol, total estriol, progesterone, prolactin, sex hormone binding globulin (SHBG), testosterone, and the like.

Analytes to determine blood disorders include, for example, B12, ferritin, folate, haptoglobin, and transferrin.

Analytes used to determine cardiac disorders include, for example, carcinoembryonic antigen (CEA), C-reactive protein (CRP), highly sensitive C-reactive protein (hsCRP), creatine kinase (CK), CK-MB, myoglobin, troponin I and T, B-type natriuretic peptide (BNP), apolipoprotein Al, apolipoprotein B, D-dimer and high density lipoprotein (HDL).

Cancer analytes include, for example, prostate specific antigen (PSA), free PSA, total PSA, fecal occult blood (FOB), acid phosphatase, alphafetoprotein (AFP), beta2 microglobulin, CA 125™, CA 15-3™, CA 19-9™, carcino embryonic antigen (CEA), PAP, pepsinogen, sqaumous cell carcinomas (SCC), and the like.

Analytes associated with inflammation and immunology include, for example, C3, C4, CRP, IgA, IgG, IgM, RF, and the like.

Analytes used to determine exposure to disease causative organisms include, for instance, rubella IgG, rubella IgM, Toxoplasmosis IgG and IgM, cytomeglovirus (CMV) IgG and IgM, HTLV III, Anti-EBNA, mononucleosis, HAA, herpes, and anti-Streptolysin O.

Infectious disease analytes include microorganisms such as Streptococcus pyogenes, Staphylococcus aureous A, Chlamydia, Syphillis, Gonococcus, Helicobactor pylori (H. pylori). Additionally, disease analytes include viral organisms such as hepatitis (HBViAb, HBsAg, HbeAb, HCV, HAA), hepatitis A virus, hepatitis B virus, IgE, cytomeglovirus (CMV), herpes viruses, rubella viruses and the like. Further included, are, for example, toxoplasmosis, anti HTLV -I/HTLV-II, BSE, Chagas antibody, CMV AB, CMV IgG, CMV IgM, CSF glucose, CSF protein, HIV AB HIV-I/HIV-2 (rDNA), rotazyme II, and the like.

Analytes pertaining to endocrinology include, for example, thyroglobulin autoantibodies (anti-Tg), thyroid peroxidase autoantibodies (anti-TPO), C-Peptide, Cortisol, HbA l c (glycosylated hemoglobin fraction), PTH, Triiodothyronine (T3), free T3, total T3, thyroid hormone, Thyroxine (T4), free T4, total T4, thyroid stimulating hormone (TSH), and the like.

Pancreatic analytes include, for example, amylase, lipase, and the like.

Veterinary analytes include, for example, Heartworm Ag, E. canis Ab, Lyme Ab, Giardia, parvovirus, FIV, FeLV, and the like.

Analytes can also include, for example, insulin, gamma globins, allergens, cystic fibrosis, toxins, such as those associated with tetanus and animal venoms, and insecticides.

The precise nature of a number of analytes together with a number of examples thereof are disclosed in U.S. Pat. Nos. 4,299,916, Nov. 10, 1981, and 4,366,241, Dec. 28, 1982, each of which is hereby incorporated by reference in its entirety.

By “porous” is meant that the matrix is composed of a material into which fluids can flow and can easily pass through, such as by lateral flow. Representative materials useful in practicing the invention described herein, include nylon, plastic, fiber containing paper, such as filter paper, chromatographic paper, and the like, nitrocellulose, glass fibers, polysullfone, polyvinylidene difluoride, polyurethane, and other porous polymers, polysaccharides, (e.g., cellulose materials, such as paper and cellulose acetate), silica, inorganic materials, such as deactivated alumina, diatomaceous earth, MgSO₄, or other inorganic finely divided material conveniently substantially uniformly dispersed in a porous polymer matrix, with polymers such as vinyl chloride, vinyl chloride-propylene copolymer, and vinyl chloride-vinyl acetate copolymer; cloth, both naturally occurring e.g., cotton and synthetic (e.g., nylon cloth), porous gels, (e.g., silica gel, agarose, dextran, and gelatin), polymeric films, (e.g., polyacrylamide), and the like. For example, the porous material can comprise Porex Chemistry A and/or Chemistry K membranes (Porex Corporation, Fairbum, Ga.) and/or novylon membrane (Cuno Incorporated, Meriden, Conn.). One skilled in the art will be aware of other porous materials that allow lateral flow.

As used herein, the term “sample pad” means the portion of the assay device which is in direct contact with the liquid sample first during test operation, i.e., it receives the sample to be tested for the analyte in question. The sample pad is made of porous material, such as porous paper, cotton, cellulose, mixed fibers, glass fiber, and the like, such that the liquid sample can migrate, via fluid flow, from the sample pad toward the absorbent pad. The sample pad can be in lateral fluid flow contact with the conjugate pad by either an overlap or end-to-end connection.

The term “conjugate pad” as used herein refers to the portion of the assay device which is in fluid flow contact with the porous material of sample pad and the novel diffusive interrupting pad. The contact can be an overlap or end-to-end connection, such that the liquid sample migrates via wicking action or by capillary forces from the sample pad through the conjugate pad to the interrupting pad. The conjugate pad comprises a porous material and a mobilizable labeled reagent that is capable of binding the analyte in question to form a labeled reagent-analyte complex which then migrates via fluid flow with the liquid sample to the interrupting pad.

The term “mobilizable” as referred to herein means diffusively or non-diffusively attached, or impregnated. The mobilizable reagents are capable of dispersing with the liquid sample and carried by the liquid sample in the fluid flow.

The term “labeled reagent” as used herein refers to any particle, protein or molecule which recognizes or binds to the analyte in question or a particle, molecule, protein which does not recognize, or bind to the analyte and has attached, conjugated or bound to it, either chemically, covalently or noncovalently, ionicly or nonionicly any substance capable of producing a signal that is detectable by visual or instrumental means. Such labels include, for example, but not limited to, chromogens, catalysts, fluorescent compounds, colloidal metallic and nonmetallic particles, dye particles, enzymes or substrates, organic polymers, latex particles, liposomes, and the like. The particle or molecule recognizing the analyte can be either natural or non-natural, and in some embodiments is a monoclonal or polyclonal antibody.

Metal sols are disclosed in U.S. Pat. Nos. 4,313,734, Feb. 2, 1982, 4,775,636, Oct. 4, 1988, each of which is hereby incorporated by reference in its entirety, and comprise a metal, a metal compound, such as metal oxides, metal hydroxides and metal salts, or polymer nuclei coated with a metal or metal compound. The metal sols can comprise, for example, metals such as platinum, gold, silver and copper. Alternatively, or additionally, the metal sols can comprise metal compounds, such as, for example, silver iodide, silver bromide, copper hydrous oxide, iron oxide, iron hydroxide or hydrous oxide, aluminum hydroxide or hydrous oxide, chromium hydroxide or hydrous oxide, vanadium oxide, arsenic sulphide, manganese hydroxide, lead sulphide, mercury sulphide, barium sulphate and titanium dioxide.

Nonmetal sols, such as carbon sols and their use are described in U.S. Pat. No. 5,559,041, Sep. 24, 1996, which is hereby incorporated by reference in its entirety. Nonmetal colloidal particles, such as selenium particles, are disclosed in U.S. Pat. No. 4,954,452, Sep. 4, 1990, which is hereby incorporated by reference in its entirety. Other nonmetals that can be used include elements within Group VI A. of the Periodic Table, sulfur, and tellurium.

Labels can also be formed from dye polymers, whereby dye molecules, or chromogenic monomers, are polymerized to form a colored polymer particle. Examples of such dyes include Congo red, Trypan blue, and Lissamine blue.

Organic polymer latex particles are disclosed in U.S. Pat. No. 5,252,459, Oct. 12, 1993, which is hereby incorporated by reference in its entirety. Such particles can comprise a plurality of non-chromophoric monomers.

Particulate labels comprising a dye or other colored substance enclosed in liposome sacs are described in U.S. Pat. Nos. 4,703,017, Oct. 27, 1987, and 5,591,645, Jan. 7, 1997, each of which is hereby incorporated by reference in its entirety.

The term “diffusive pad”, “interrupting pad” or “diffusive interrupting pad” as used herein refers to the portion of the assay device which is in fluid flow contact with the porous material of the conjugate pad and the reaction membrane, or in some embodiments, in fluid flow contact with the sample pad and conjugate pad. Therefore, the diffusive pad or interrupting pad can be a single pad in a single location or multiple pads in multiple locations. The contact or contacts can be an overlap connection, such that the analyte in the labeled reagent-analyte complex, unbound labeled reagent, and unbound analyte migrates via wicking action with the liquid sample from the conjugate pad through the diffusive interrupting pad to the reaction membrane. The diffusive interrupting pad comprises a porous material, for example, fiber glass. The diffusive interrupting pad can further comprise chemicals to increase the flow affinity and/or its uniformity, therefore increase binding sensitivity and specificity, and hence accuracy of the labeled reagent and analyte in question such that the signal provided at the reaction membrane is enhanced. As mentioned above, the “diffusive pad” or “interrupting pad” was/is not a part of the prior art of any existing lateral flow rapid test devices, or any similar prior art, before this invention.

The term “reaction membrane” as used herein refers to the portion of the assay device which is in fluid flow contact with the porous material of the diffusive interrupting pad and the absorbent pad. The contact can be an overlap or end-to-end connection, such that the labeled reagent-analyte complex, unbound labeled reagent, and unbound analyte migrate through wicking action, with the liquid sample from the interrupting pad through the reaction membrane to the absorbent pad. The reaction membrane comprises a porous material, for example, nitrocellulose, and immobilized capture reagent(s). The reaction membrane optionally further comprises an immobilized control reagent, as an internal system control indicator.

The term “immobilized” as used herein refers to reagents which are attached to the support material such that fluid flow of the liquid sample does not affect the placement of the immobilized reagents in the porous material. Such attachment can be through various methods, such as covalent or ionic means. Those skilled in the art will be aware of means of attachment to immobilize various particles.

The term “capture reagent” as used herein refers to any particle or molecule which recognizes or binds the analyte in question. The capture reagent is capable of forming an immuno-sandwich binding-complex with the labeled reagent-analyte complex. In some embodiments, the capture reagent is immobilized to the porous material of the reaction membrane. The capture reagent is not affected by the fluid flow of the liquid sample due to its firm immobilization to the porous material. The particle or molecule can be natural, or non-natural, i.e., synthetic. Once the capture reagent binds the labeled reagent-analyte complex it prevents the complex from continuing with the fluid flow of the liquid sample, and finally establishes a visible colored test line to indicate a positive or a negative result.

The term “control reagent” as used herein refers to any particle or molecule which is capable of binding any labeled accordant reagent that does not recognize or bind the analyte of question in the sample. The control reagent can be immobilized to the porous material of the reaction membrane downstream of the capture reagent. Once it binds the accordant unbound labeled reagent it immobilizes the labeled reagent and prevents it from continuing fluid flow with the liquid sample to internally indicate the correct function of this lateral flow testing system.

The term “absorbent pad” as used herein refers to the portion of the assay device which is in fluid flow contact with the reaction membrane, which is capable of absorbing excess liquid sample to prevent any back flow of flow fluid during operating time. The contact with the reaction membrane can be either by overlap or end-to-end connection. The absorbent pad comprises porous material, such as porous paper and the like.

While the preferred embodiment of the invention has been described, modifications can be made and other embodiments may be devised without departing from the spirit of the invention and the scope of the appended claims.

What is claimed is:

1. In a lateral flow chromatographic test apparatus comprising a conjugate pad adapted to carry a soluble binding member molecule specific to a particular analyte and a reaction membrane carrying a capture binding member molecule, an improvement which comprises:
a first porous interrupting diffusive pad located between a portion of said conjugate pad and a portion of said reaction membrane, thereby creating a plurality of convergent fluid paths between said conjugate pad and said membrane.

2. The improvement of claim 1, wherein said portion of said conjugate pad overlaps said first diffusive pad, and a portion of said first diffusive pad overlaps said portion of said reaction membrane.

3. The improvement of claim 1, wherein said first diffusive pad is pretreated with a surfactant.

4. The improvement of claim 3, wherein said surfactant comprises a non-ionic detergent.

5. The improvement of claim 1, wherein said first diffusive pad has a first pretreatment condition.

6. The improvement of claim 5, wherein said first pretreatment condition is being pretreated with a surfactant.

7. The improvement of claim 5, wherein said first pretreatment condition is selected from the group consisting of: being pretreated with a surfactant; being pretreated with a pH conditioner, being pretreated with a non-specific adhesive blocking molecule, and having no pretreatment.

8. The improvement of claim 1, wherein said improvement further comprises a second diffusive pad, wherein said first diffusive pad has a first pretreatment condition and said second diffusive pad has a second pretreatment condition, and wherein said first pretreatment condition is different from said second pretreatment condition.

9. The improvement of claim 8, wherein said first pretreatment condition is selected from the group consisting of: being pretreated with a surfactant; being pretreated with a pH conditioner, being pretreated with a non-specific adhesive blocking molecule, and having no pretreatment.

10. The improvement of claim 1, wherein said diffusive pad is made from material having a plurality of intersecting surface structures, wherein a junction between first and second of said structures is not substantially parallel.

11. The improvement of claim 1, wherein said diffusive pad comprises a material selected from the group consisting of: fiberglass, cellulose and fibrous plastic and the like.

12. The improvement of claim 1, wherein said diffusive pad comprises means for evening a fluid front passing therethrough.

13. The improvement of claim 1, wherein there is no direct fluid flow contact between said conjugate pad and said reaction membrane.

14. A method for conducting a lateral flow immunoassay test comprises:
diffusing a fluid flow from a conjugate pad through a diffusive pad before said flow reaches a reaction membrane.

15. A method for detecting a biological condition by means of a lateral flow chromatographic immunoassay rapid test on a sample of a patient's body fluid which comprises:
exposing said sample to a strip having at least one line carrying an immunoassay complex component responsive to at least one inter-reactive component indicative of the presence of said condition in said patient;
mixing said sample after said exposing, using a diffusive pad, to form a mixture; and,
distributing said mixture to a reaction membrane which carries said at least one line.

16. The method of claim 15, wherein said mixing comprises evening a fluid front within said diffusive pad.

17. The method of claim 15, which further comprises pretreating said diffusive pad with a surfactant.

18. The method of claim 15, wherein said detecting occurs at an accuracy of at least 99.5%.
